(19) United States
(12) Patent Application Publication
(10) Pub. No.: US 2023/0183750 A1
(43) Pub. Date: Jun. 15, 2023

(54) HDR ENHANCERS

(71) Applicants: OXFORD GENETICS LIMITED, Oxfordshire (GB); OXFORD UNIVERSITY INNOVATION LIMITED, Oxford (GB)

(72) Inventors: Ryan Cawood, Oxfordshire (GB); Tom Payne, Oxfordshire (GB); Suzanne Snellenberg, Oxfordshire (GB); Abhishek Srivastava, Oxfordshire (GB)

(21) Appl. No.: 17/999,307

(22) PCT Filed: May 20, 2021
(86) PCT No.: PCT/GB2021/051215

§ 371 Date: Nov. 18, 2022

(30) Foreign Application Priority Data
May 21, 2020 (GB) 2007577.6
Feb. 15, 2021 (GB) 2102063.1

(51) Int. Cl. C12N 15/90; C12N 15/11; C12N 9/22

(52) U.S. Cl. CPC C12N 15/907; C12N 15/11; C12N 9/22; C12N 2310/20; C12N 2800/80

(57) ABSTRACT

The invention relates to a method for promoting the modification, preferably by homology-dependent repair (HDR), of a target site in a genome of a cell. The method comprises the steps of introducing a template DNA molecule and one or more DNA repair inhibitors into a cell which comprises or is capable of expressing a site-specific DNA endonuclease (e.g. Cas9). The DNA repair inhibitors comprise one or more aurora kinase inhibitors, wherein the aurora kinase inhibitors are selected from the group consisting of AT9283, PHA-680632, TAK-901 and CCT137690, together with one or more other inhibitors. The invention also relates to kits comprising the aforementioned DNA repair inhibitors.

### CROSS-REFERENCE

This application is a 371 U.S. national phase of PCT/GB2021/051215, filed May 20, 2021, which claims priority from GB 2007577.6, filed May 21, 2020 and GB 2102063.1, filed Feb. 15, 2021, all which are incorporated by reference in their entirety.

### FIELD OF THE INVENTION

The invention relates to a method for promoting the modification, preferably by homology-dependent repair (HDR), of a target site in a genome of a cell. The method comprises the steps of introducing a template DNA molecule and one or more DNA repair inhibitors into a cell which comprises or is capable of expressing a site-specific DNA endonuclease (e.g. Cas9). The DNA repair inhibitors comprise one or more aurora kinase inhibitors, wherein the aurora kinase inhibitors are selected from the group consisting of AT9283, PHA-680632, TAK-901 and CCT137690, together with one or more other inhibitors. The invention also relates to kits comprising the aforementioned DNA repair inhibitors.

### BACKGROUND OF THE INVENTION

Genetically-engineered cellular and animal models are an important tool for research and development of novel therapeutics. The discovery and development of gene-editing tools such as CRISPR/Cas9, which can precisely modify the genome, has revolutionized this field. It has also helped to establish new diseases models and to accelerate drug development in recent years.

CRISPR/Cas9 recognizes specific DNA sequences with a 3′ “NGG” (the PAM site) in the genome; it introduces double-stranded breaks (DSBs) in a precise and efficient manner. These double-stranded breaks initiate a DNA damage response in the cell and they are repaired by one of two competitive pathways: non-homologous end joining (NHEJ) or homology-dependent repair (HDR, also known as homology-directed repair). The NHEJ pathway involves random insertion or deletions (indels) at the site of DNA damage, while the HDR pathway enables more precise modification, but it requires a homologous donor template for the repair.

In NHEJ, Ku70/Ku80 proteins first bind to the exposed DNA end at the cut site as a heterodimer and then they recruit DNA protein kinase catalytic subunits (DNA-PKcs). Binding of the Ku70/80 heterodimer and DNA-PKcs initiates the recruitment of various other effector proteins of the NHEJ pathway such as XLF and XRCC4, and the DNA break is then repaired by ligation mediated by DNA ligase IV.

In the absence of the classical NHEJ pathway, the Alternative NHEJ (Alt-NHEJ) pathway gets activated, which is independent of Ku70 and Ku80 proteins; this depends on PARP1 and PARP2. PARP1/2 recruit a different set of effector proteins such as XRCC1 to the site of DNA damage, and the DNA break is then sealed by DNA ligase Ill.

In the presence of a donor molecule, DSB repair can proceed by the HDR pathway. This starts with the binding of an MRE11-Rad50-NBS1 (MRN) complex at DSB site, followed by exonuclease activity of CUP to generate long 3′ ssDNA overhangs on either side of DNA damage. These ssDNAs are stabilized by binding of replication protein A (RPA) and followed by the action of rad51 and rad52 proteins which help in donor template annealing and the precise repair of the DSBs.

Although precise, DSB repair by the HDR pathway is not very efficient. Furthermore, it depends on factors such as cell cycle stage (S and G2 phase), availability of donor template and accessory proteins.

In order to achieve gene correction via HDR, the cells must either be in S-phase where HDR is preferred over NHEJ, or the cell must exhaust all its NHEJ-like repair options before resorting to HDR. Different approaches have been reported to improve the HDR efficiency to increase the precise genome engineering: these include nucleofection, cell cycle synchronization to S-phase, use of small molecules (for example inhibitors of proteins involved in NHEJ) and tethering donor molecule to nucleases. However, each of these options have specific limitations.

It has been suggested that inhibition of competing pathways could increase HDR. This has been shown by inhibiting the proteins involved in NHEJ pathway: for example, inhibition of DNA-PKs by NU7441 and NU7026; inhibition of Ku70/80 by KU-0060648; and inhibition of DNA ligase IV by SCR7. However, these observations vary in different cell lines and depend on the gene targeted.

It has been known that the pathway choice is largely determined at the very early stages of DSBs by the competition between the 53BP1 and BRCA1 regulatory proteins, triggering either the protection or resection of DSB ends, which results in activation of the NHEJ or HDR pathway, respectively. 53BP1 blocks end resection (Bunting et al., 2010), and thus inhibits BRCA1 accumulation (Escribano-Diaz et al., 2013; Zimmermann et al., 2013).

53BP1 is recruited to DSBs by recognition of the Ubiquitin mark at Lysine 15 of histone H2A (H2A15Ub) (Fradet-Turcotte et al., 2013) and dimethylation at lysine 20 of histone H4 (H4K20me2) in chromatin. The HDR pathway requires the dislocation of 53BP1 and the resection of DSB ends in order to initiate BRCA 1 accumulation. During the SIG2 phase, BRCA1 recruits CtIP and the MRN complex. This complex initiates a cleavage step which is then further resected at the 5′ end by Exo1 (Sartori et al., 2007; Symington and Gautier, 2011; Symington, 2016) extending on each side of the DSB (Zakharyevich et al., 2010). The exposed single-stranded DNA (ssDNA) is protected by binding of RPA1 that is subsequently replaced by Rad51 through the action of BRCA2 and Rad52, forming a nucleo-filament competent for homology search and strand invasion for HDR based DSB repair.

This suggests that histone modifications such as methylation and ubiquitination are involved in regulating the recruitment and retention of 53BP1, which in turn decides the dynamics of NHEJ vs HDR. These modifications of histones are part of epigenetic mechanisms.

### SUMMARY OF THE INVENTION

The invention relates to a method for promoting the modification, preferably by homology-dependent repair (HDR), of a target site in a genome of a cell. The method comprises the steps of introducing a template DNA molecule and one or more DNA repair inhibitors into a cell which comprises or is capable of expressing a site-specific DNA endonuclease (e.g. Cas9). The DNA repair inhibitors comprise one or more aurora kinase inhibitors, wherein the aurora kinase inhibitors are selected from the group consisting of AT9283, PHA-680632, TAK-901 and CCT137690, together with one or more other inhibitors. The invention also relates to kits comprising the aforementioned DNA repair inhibitors.

### DESCRIPTION OF THE DRAWINGS

FIG. 1 shows a schematic diagram of the HEK293 reporter cell line for the HDR assays.

FIG. 2A is a schematic diagram of HDR assay using wtCas9.

FIG. 2B is a representative FACS profile of HDR assay using wtCas9.

FIG. 3 shows dose dependency of top small molecule hits identified with Cas9.

FIG. 4 shows small molecule combinations using Cas9.

FIG. 5 shows the effect of different aurora kinase inhibitors on HDR efficacy.

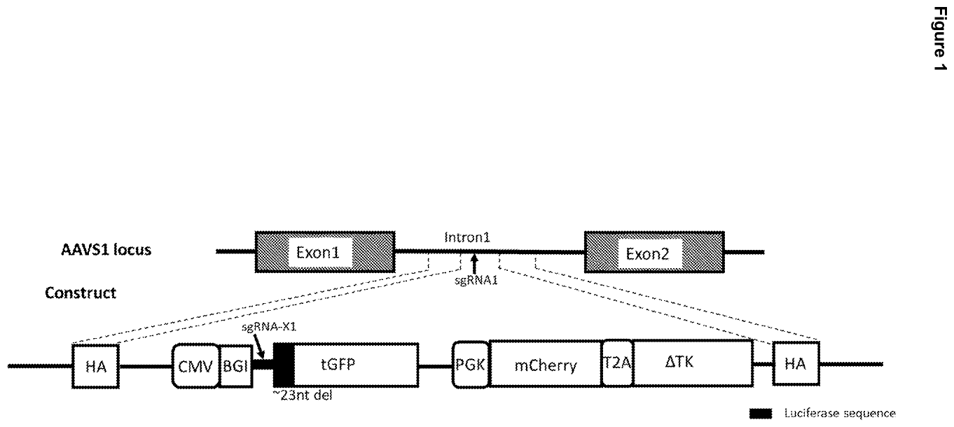

### DETAILED DESCRIPTION OF THE INVENTION

Inhibitor compounds have now been identified which inhibit epigenetic modifications; their effects on HDR efficiency have been monitored in a reporter cell line. These compounds can be used to increase HDR efficiency when DSBs are generated by nucleases such as Cas9. A number of the compounds have not previously been reported to be associated with increasing HDR efficiency.

These compounds are usable in a number of different cell lines.

The compounds have been found to increase the DNA repair efficiency in the experiments described in the Examples herein, either alone or in combinations with other inhibitor compounds.

In one embodiment, the invention provides a method for promoting the modification of a target site in a genome of a cell, the method comprising the steps of introducing:
(i) a template DNA molecule which has DNA sequence homology with the target site; and
(ii) one or more inhibitors;

into a cell which comprises or is capable of expressing a site-specific DNA endonuclease, thereby promoting the site-specific cleavage of the cell genome by the site-specific DNA endonuclease and the modification, of the target site in the cell genome, characterised in that the one or more inhibitors comprise one or more aurora kinase inhibitors selected from the group consisting of AT9283, PHA-680632, TAK-901 and CCT137690, and the site-specific endonuclease is one which produces a blunt-end double-stranded cut in the cell genome.

The one or more inhibitors may comprise AT9283 and/or PHA-680632.

In another embodiment, the invention provides a method for promoting the modification of a target site in a genome of a cell, the method comprising the steps of introducing:
(i) a template DNA molecule which has DNA sequence homology with the target site; and
(ii) one or more inhibitors;

into a cell which comprises or is capable of expressing a site-specific DNA endonuclease, thereby promoting the site-specific cleavage of the cell genome by the site-specific DNA endonuclease and the modification, of the target site in the cell genome, characterised in that the one or more inhibitors comprise AT9283, and the site-specific endonuclease is one which produces a blunt-end double-stranded cut in the cell genome.

The invention provides a method for promoting the modification of a target site in a genome of a cell. The method of the invention may be carried out in vivo, ex vivo or in vitro, preferably in vitro.

The site-specific DNA endonuclease will cut the DNA at or in the vicinity of the target site, thus allowing the DNA sequence at the target site to be modified (preferably by homology-directed repair), utilising the template DNA molecule as a template for the repair.

The modification of the target site may be the insertion, deletion or substitution of one or more nucleotides in the genome of the cell.

The method of the invention utilises one or more site-specific DNA endonucleases.

Each site-specific DNA endonuclease may be present in the cell in the form of a polypeptide (e.g. Cas9) or a rbonudeoprotein particle (e.g. Cas9/gRNA).

In some embodiments, the cell is one which is expressing or capable of expressing one or more site-specific DNA endonucleases. For example, a nucleic acid molecule encoding a site-specific DNA endonuclease may be integrated into a cellular genome (e.g. nuclear genome); the cell may comprise a plasmid or vector encoding a site-specific DNA endonuclease; or the cell may comprise a virus particle (e.g. adenovirus, adeno-associated virus, lentivirus) encoding a site-specific DNA endonuclease.

The DNA plasmid or vector or virus may additionally comprise suitable regulatory elements (e.g. an enhancer, a promoter, a terminator) which are operably-associated with the nucleotide sequence which encodes the site-specific DNA endonuclease in order to control expression of that endonuclease. The DNA plasmid or vector may additionally comprise a selection gene, e.g. for antibiotic resistance.

More than one endonuclease may be encoded by the same DNA plasmid, vector or virus.

In some embodiments, the cell comprises a nucleic acid molecule encoding a site-specific DNA endonuclease, wherein the expression of the site-specific DNA endonuclease is under the control of an inducible promoter. The method of the invention may additionally comprise the step of inducing the expression of the site-specific DNA endonuclease.

In some embodiments, the method additionally comprises the step of introducing one or more site-specific DNA endonucleases into the cell.

The cell may comprise, express or be capable of expressing one or more site-specific DNA endonucleases, e.g. 1, 2, 3 or 4 site-specific DNA endonucleases.

The endonuclease is a site-specific endonuclease, i.e. it is capable of targeting one site or a plurality of sites in the cell genome based on the nucleotide sequence of that site or sites.

The endonuclease is capable of making blunt-end double-stranded cuts within DNA molecules, i.e. within a cell genome.

The endonuclease may be RNA-guided (e.g. CRISPR/Cas9) or non-RNA-guided (e.g. zinc finger nuclease or TALENs).

Preferably, the endonuclease is a RNA-guided endonuclease. More preferably, the endonuclease is a CRISPR RNA-guided endonuclease. CRISPR is an acronym for Clustered, Regularly Interspaced, Short, Palindromic Repeats. The CRISPR endonuclease is one which is capable of forming a complex with a CRISPR guide RNA (e.g. a crRNA-tracrRNA), preferably with a CRISPR single guide RNA (sgRNA). The CRISPR endonuclease is one which, when complexed with a CRISPR RNA, is capable of targeting the thus-produced complex to a target site in the cell genome which has a nucleotide sequence which is complementary to that of the spacer element in the guide RNA. In some embodiments, the nucleotide sequence encoding the CRISPR endonuclease is codon-optimized for expression in the target cell.

In some embodiments, the CRISPR endonuclease is a Type II CRISPR system enzyme. In some embodiments, the CRISPR endonuclease is Cas9 or a Cas9-like polypeptide which produces a blunt end cut. In some embodiments, the Cas9 endonuclease is derived from S. pneumoniae, S. pyogenes, or S. thermophilus Cas9, or a variant thereof. Preferably, the CRISPR endonuclease is a wild-type Cas9, e.g. SpCas9.

If the endonuclease is a RNA-guided endonuclease, then one or more cognate CRISPR guide RNAs will also need to be introduced into the cell or be present within the cell.

A cognate CRISPR gRNA is one which, when complexed with a CRISPR endonuclease, is capable of targeting the thus-produced gRNA/CRISPR endonuclease complex to a target site in the cell genome which has a nucleotide sequence which is complementary to that of the target/guide element in the gRNA.

The CRISPR gRNA is preferably a single guide RNA (sgRNA). In other embodiments, a dual RNA (crRNA+tracrRNA) may be used. The RNA is made up of ribonucleotides A, G, T and U. Modified ribonucleotides may also be used, e.g. to increase the stability of the RNA.

A sgRNA is a chimeric RNA which replaces the crRNA/tracrRNA which are used in the native CRISPR/Cas systems (e.g. Jinek, M. et al. (2012), “A programmable dual-RNA-guided DNA endonuclease in adaptive bacterial immunity”, Science 337, 816-821). The term sgRNA is well accepted in the art. The sgRNA comprises a spacer element. The spacer element is also known as a spacer segment or guide sequence. The terms spacer element, spacer segment and guide sequence are used interchangeably herein. The sgRNA comprises a region which is capable of forming a complex with a CRISPR enzyme, e.g. a CRISPR endonuclease, e.g. Cas9. The sgRNA comprises, from 5′ to 3′, a spacer element which is programmable (i.e. the sequence may be changed to target a complementary DNA target site), followed by the sgRNA scaffold. The sgRNA scaffold may technically be divided further into modules whose names and coordinates are well known in the art (e.g. Briner, A. E. et al. (2014). “Guide RNA functional modules direct cas9 activity and orthogonality”. Molecular Cell, 56(2), 333-339).

Targeted DSBs introduced by CRISPR/Cas system require a PAM (e.g. NGG) recognition sequence. The CRISPR RNA-guided endonuclease may be one which recognises a non-native PAM sequence.

Guide RNAs, when required, may be introduced into the cell by any suitable method, e.g. by electroporation, nucleofection or lipofection.

In some embodiments, the nuclease is a non-RNA-guided nuclease, e.g. a zinc finger nuclease or TALENs. Zinc-finger nucleases (ZFNs) are artificial restriction enzymes generated by fusing a zinc finger DNA-binding domain to a DNA-cleavage domain. Transcription activator-like effector nucleases (TALENs) comprise TAL-effector domains fused to a nuclease domain. ZFNs and TALENs have been successfully used for genome modification in a variety of different species. See, for example, U.S. Pat. Nos. 7,888,121; 8,409,861; 8,586,526; 7,951,925; 8,110,379; 7,919,313; 8,597,912; 8,153,399; 8,399,218; and US Patent Publications 2009/0203140; 2010/0291048; 2010/0218264; and 2011/0041195.

The method of the invention encompasses introducing into the cell one or more inhibitors, preferably inhibitors of one or more of the cell's proteins which are involved—directly or indirectly—in the repair of double-stranded breaks. The proteins to be inhibited are preferably ones which are involved in one or more of the NHEJ (classical-NHEJ and alternative-NHEJ) repair pathways. These are proteins are endogenously present within the cell. One or more of the cell's proteins may be inhibited.

In some embodiments, the proteins involved in the repair of double-stranded breaks are proteins involved in:
(a) the classic DNA-PKcs dependent NHEJ (“error-free”) pathway;
(b) the PARP1/2 dependent alternative NHEJ pathway; or
(c) the PARP1/2 dependent SSB repair pathway.

It will be appreciated that the inhibitors are not ones which are significantly toxic to the cell, i.e. inhibitors which lead to significant amounts of cell death. As used herein, the term “significantly toxic” refers to a concentration of the inhibitor(s) which leads to more than 30%, 35%, 40% or 50% cell death when incubated in tissue culture media with HEK293 cells at 37° C. in a CO₂ incubator for 24 hours; and then in tissue culture media without the inhibitor(s) for a further 48 hours.

Preferably, the HDR efficiency is at least 6%, 8% or 10%; more preferably at least 12%, 14%, 16%, 18% or 20%. HDR efficiency may be assayed by fluorescence using FACS (if fluorescence-based reporter cell lines are used) or luminescence by plate reader (if luminescence-based reporter cell lines are used). Alternatively, a PCR-based approach may be used where PCR-amplified target samples are sequenced by Sanger sequencing or amplicon sequencing (e.g. NGS), and the results are analysed by suitable bioinformatics tools such as TIDE or ICE.

More specifically, an HDR assay using a HEK reporter cell line containing truncated EGFP may be used. These cells may be transfected with a transfection complex containing the CRISPR endonuclease and a donor sequence. Cas9 RNPs may be prepared by following the manufacturers' guidelines. The transfection complex may be prepared by adding Cas9 RNP or Cas9 nickase RNP, along with ssOligo donor and lipofectamine 2000 in Optimem. Reagents should be mixed well and incubated for 20 mins. After 20 mins, 50 μl of transfection complex (at an optimal concentration—see the Examples herein) may be transferred in a 96 well plate and 50 μl HEK293 reporter cell line suspension (9×10⁵ cells/ml) added followed by 50 μl of cell culture medium containing appropriate concentration(s) of inhibitor(s). Cells may be incubated at 37° C. in a CO₂ incubator for 24 hours and then inhibitor-containing media should be replaced with fresh media without inhibitor. Cells are then further incubated for 48 hours. After 48 hours, cells may be trypsinized and resuspended in PBS containing 10% FBS. Samples may be run on FACS and the percentage of EGFP in the population measured. Presence of EGFP directly correlates with HDR efficiency.

In one embodiment, the one or more inhibitors comprise AT9283 and the site-specific endonuclease is one which produces a blunt-end double-stranded cut in the cell genome (preferably Cas9).

AT9283 (CAS No. 896466-04-9) is a JAK2/3 inhibitor and/or also inhibits aurora A/B kinase. It has the following structure:

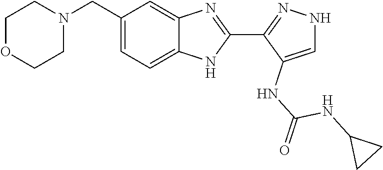 [SMILES: O=C(Nc1c[nH]nc1-c1nc2cc(CN3CCOCC3)ccc2[nH]1)NC1CC1]

The invention also extends to variants or derivatives of AT9283 which are also JAK2/3 inhibitors and/or aurora kinase inhibitors.

PHA-680632 (CAS No. 398493-79-3) inhibits aurora A/B/C kinases. It has the following structure:

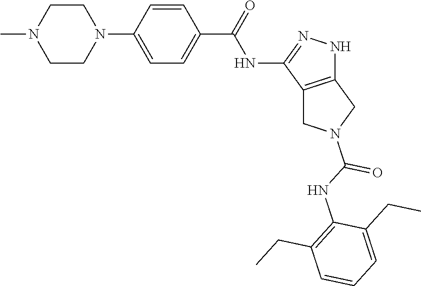 [SMILES: CCc1cccc(CC)c1NC(=O)N1Cc2[nH]nc(NC(=O)c3ccc(N4CCN(C)CC4)cc3)c2C1]

The invention also extends to variants or derivatives of PHA-680632 which are also aurora kinase inhibitors.

TAK-901 (CAS No. 934541-31-8) is an aurora NB kinase inhibitor and a JAK inhibitor. It has the following structure:

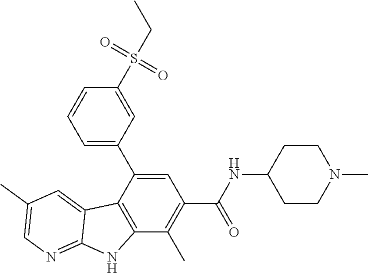

The invention also extends to variants or derivatives of TAK-901 which are also aurora kinase inhibitors and/or JAK inhibitors.

CCT137690 (CAS No. 1095382-05-0) is an aurora A/B/C kinase inhibitor; it also inhibits receptor tyrosine kinase FLT3. It has the following structure:

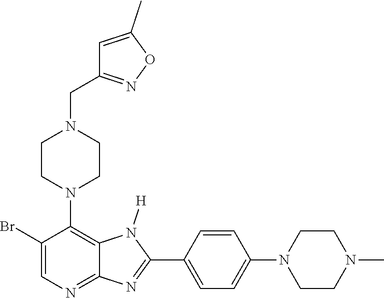 [SMILES: Cc1cc(CN2CCN(c3c(Br)cnc4nc(-c5ccc(N6CCN(C)CC6)cc5)[nH]c34)CC2)no1]

The invention also extends to variants or derivatives of CCT137690 which are also aurora kinase inhibitors and/or FLT3 inhibitors.

In other preferred aspects of this embodiment, the one or more inhibitors comprise AT9283 and/or PHA-680632 together with one or more additional inhibitors selected from the group consisting of NU7441, S-PFI-2 and SMI-4a. The group may also comprise NU7026.

In other preferred aspects of this embodiment, the one or more inhibitors comprise TAK-901 and/or CCT137690 together with one or more additional inhibitors selected from the group consisting of NU7441, S-PFI-2 and SMI-4a. The group may also comprise NU7026.

NU7441 (CAS No. 503468-95-9) is a DNA-dependent protein kinase inhibitor. It has the following structure:

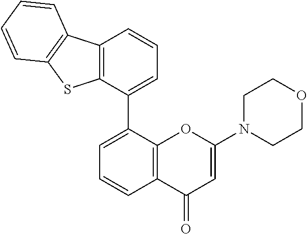 [SMILES: O=c1cc(N2CCOCC2)oc2c(-c3cccc4c3sc3ccccc34)cccc12]

The invention also extends to variants or derivatives of NU7441 which are also DNA-dependent protein kinase inhibitors.

NU7026 (CAS No. 154447-35-5) is a DNA-dependent protein kinase inhibitor. It has the following structure:

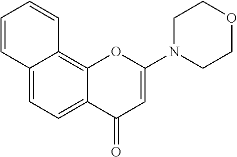 [SMILES: O=c1cc(N2CCOCC2)oc2c1ccc1ccccc12]

The invention also extends to variants or derivatives of NU7026 which are also DNA-dependent protein kinase inhibitors.

S-PFI-2 (CAS No. 1627607-88-8) is a SETD7 inhibitor. It has the following structure:

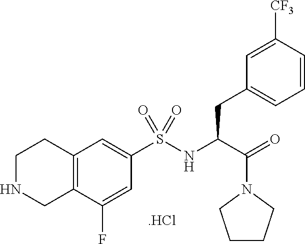 [SMILES: O=C([C@H](Cc1cccc(C(F)(F)F)c1)NS(=O)(=O)c1cc(F)c2c(c1)CCNC2)N1CCCC1 ; ".HCl"]

The invention extends to variants or derivatives of S-PFI-2 which are also methyltransferase inhibitors.

SMI-4a (CAS No. 438190-29-5) is a Pim1 and Pim2 inhibitor. It has the following structure:

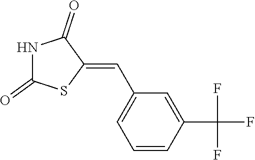 [SMILES: O=C1NC(=O)/C(=C/c2cccc(C(F)(F)F)c2)S1]

The invention extends to variants or derivatives of SMI-4a which are also Pim1 and/or Pim2 inhibitors.

In one preferred embodiment, the one or more inhibitors comprise AT9283, together with NU7441 and/or NU7026. In other embodiments, the one or more inhibitors comprise: AT9283, NU7441 and S-PFI-2; AT9283, NU7441 and SMI-4a; AT9283, NU7441, S-PFI-2 and SMI-4a; AT9283, NU7026 and S-PFI-2; AT9283, NU7026 and SMI-4a; or AT9283, NU7026, S-PFI-2 and SMI-4a.

In another preferred embodiment, the one or more inhibitors comprise PHA-680632, together with NU7441 and/or NU7026. In another preferred embodiment, the one or more inhibitors comprise TAK-901, together with NU7441 and/or NU7026. In another preferred embodiment, the one or more inhibitors comprise CCT137690, together with NU7441 and/or NU7026.

In other embodiments, the one or more inhibitors comprise:

PHA-680632, NU7441 and S-PFI-2;

PHA-680632, NU7441 and SMI-4a;

PHA-680632, NU7441, S-PFI-2 and SMI-4a;

PHA-680632, NU7026 and S-PFI-2;

PHA-680632, NU7026 and SMI-4a; or

PHA-680632, NU7026, S-PFI-2 and SMI-4a.

In other embodiments, the one or more inhibitors comprise:

TAK-901, NU7441 and S-PFI-2;

TAK-901, NU7441 and SMI-4a;

TAK-901, NU7441, S-PFI-2 and SMI-4a;

TAK-901, NU7026 and S-PFI-2;

TAK-901, NU7026 and SMI-4a; or

TAK-901, NU7026, S-PFI-2 and SMI-4a.

In other embodiments, the one or more inhibitors comprise:

CCT137690, NU7441 and S-PFI-2;

CCT137690, NU7441 and SMI-4a;

CCT137690, NU7441, S-PFI-2 and SMI-4a;

CCT137690, NU7026 and S-PFI-2;

CCT137690, NU7026 and SMI-4a; or

CCT137690, NU7026, S-PFI-2 and SMI-4a.

In another preferred embodiment, the one or more inhibitors comprise AT9283 and PHA-680632, together with NU7441 and/or NU7026.

In another preferred embodiment, the one or more inhibitors comprise AT9283 and PHA-680632 and TAK-901, together with NU7441 and/or NU7026. In another preferred embodiment, the one or more inhibitors comprise AT9283 and PHA-680632 and CCT137690, together with NU7441 and/or NU7026.

In other embodiments, the one or more inhibitors comprise:

AT9283, PHA-680632, NU7441 and S-PFI-2;

AT9283, PHA-680632, NU7441 and SMI-4a;

AT9283, PHA-680632, NU7441, S-PFI-2 and SMI-4a;

AT9283, PHA-680632, NU7026 and S-PFI-2;

AT9283, PHA-680632, NU7026 and SMI-4a; or

AT9283, PHA-680632, NU7026, S-PFI-2 and SMI-4a;

and optionally one or both of TAK-901 and CCT137690.

In this aspect of the invention, the site-specific endonuclease is one which produces a blunt-end double-stranded cut in the cell genome. Preferably, the site-specific endonuclease is Cas9 or a variant or derivative thereof which produces a blunt-end double-stranded DNA cut.

Concentrations of the inhibitors may be selected so as to maximise the inhibitory effect of the inhibitor whilst not being significantly toxic to the cell. Preferably, the concentrations of each inhibitors are independently 0.01 μM to 50 μM, e.g. 0.01 μM to 0.5 μM, 0.5 μM to 1.0 μM, 1.0 μM to 5.0 μM or 5.0 μM to 20 μM, more preferably 0.05 μM to 20 μM, for example approximately 0.05 μM, 0.1 μM, 0.2 μM, 0.5 μM, 1.0 μM, 2.0 μM, 5.0 μM, 10 μM or 20 μM.

Preferably, the concentration of AT9283 is 0.01 μM to 0.5 μM, or 0.05 μM to 0.2 μM, more preferably 0.01 μM to 0.1 μM, and most preferably about 0.05 μM.

In some embodiments, the concentration of PHA-680632 is 0.1 μM to 5.0 μM, or 0.5 μM to 2.0 μM, more preferably 1.0 μM to 5.0 μM, and most preferably about 2.0 μM.

In some embodiments, the concentration of TAK-901 is 0.01 μM to 0.5 μM, or 0.05 μM to 0.2 μM, more preferably 0.01 μM to 0.1 μM, and most preferably about 0.1 μM.

In some embodiments, the concentration of CCT137690 is 0.01 μM to 0.5 μM, or 0.05 μM to 0.2 μM, more preferably 0.01 μM to 0.1 μM, and most preferably about 0.1 μM.

Preferably, the concentration of NU7441 is 0.1 μM to 5.0 μM, or 0.5 μM to 2.0 μM, more preferably 1.0 μM to 5.0 μM, and most preferably about 2.0 μM.

Preferably, the concentration of NU7026 is 0.1 μM to 5.0 μM, or 0.5 μM to 2.0 μM, more preferably 1.0 μM to 5.0 μM, and most preferably about 2.0 μM.

Preferably, the concentration of S-PFI-2 is 1 μM to 50 μM, or 5 μM to 20 μM, more preferably 15 μM to 25 μM, and most preferably about 20 μM.

Preferably, the concentration of SMI-4a is 1 μM to 50 μM, or 5 μM to 20 μM, more preferably 15 μM to 25 μM, and most preferably about 20 μM.

Preferably, the cells are incubated with the one or more inhibitors for 1-36 hours, more preferably 6-24 hours, and most preferably for about 18 hours.

The template DNA molecule is a DNA molecule which has DNA sequence homology with the target site. It acts as a template for the repair (preferably homology-directed repair) of the cleaved target site. The template DNA may be single-stranded or double-stranded DNA, preferably single-stranded DNA. The template DNA may be provided in the form of linear DNA or it may be expressed from a virus (e.g. adeno-associated virus or integration-deficient lentivirus). The template DNA may be introduced into the cell by any suitable means, e.g. transfection, electroporation, etc. In some embodiments, donor DNA may be introduced along with DNA endonuclease by transfection, e.g. using lipofectamine reagent, or by electroporation.

The sequence of the template DNA may or may not be based on the sequence which it is intended to replace. For example, the template DNA may have substantially the same DNA sequence as the sequence which it is intended to replace at the target site, but the template DNA may comprise mutations (e.g. a SNP, an insertion or a deletion) compared to the DNA sequence of the sequence which it is intended to replace. In other cases, for example where it is desired to delete the cellular sequence or to replace it with a different DNA, the template DNA may not have any significant degree of sequence identity with the sequence which it is intended to replace (apart from the homology arms, as discussed below).

The length of the template DNA molecule may be from 1 to 8000 nucleotides, preferably 0 to 500 nucleotides, more preferably from 0 to 200 nucleotides. The length of the template DNA depends on the desired modification to be introduced. The template DNA molecule will span the cut in the target site produced by the DNA endonuclease.

The template DNA molecule comprises homology arms, wherein the homology arms are capable of promoting the replacement of all or part of the target sequence in the cellular genome with a sequence having the sequence of the template DNA sequence. Preferably, there are two homology arms: one at the 5′ end of the template DNA molecular and one at the 3′-end of the template DNA molecule. The upstream (5′) homology arm comprises a stretch of DNA whose sequence has identity to a stretch of DNA that lies in the 5′-end of the target cellular sequence. The downstream (3′) homology arm comprises a stretch of DNA whose sequence has identity to a stretch of DNA that lies in the 3′-end of the target cellular sequence.

Preferably, the degree of sequence identity between the 5′ homology arm and the corresponding sequence in the cellular genome is at least 90%, more preferably at least 95% or 99%, or it is 100%. Preferably, the degree of sequence identity between the 3′ homology arm and the corresponding sequence in the cellular genome is at least 90%, more preferably at least 95% or 99%, or it is 100%. The homology arms may each independently be 5 to 1000 nucleotides in length, preferably 10 to 800, and more preferably independently 20 to 80 nucleotides in length.

In some embodiments, the nucleotide sequence of the target molecule comprises a sequence of a gene encoding a protein, e.g. a protein that is lacking in the cell or a corrected (wild-type) version of protein which is present in mutated form in the cell.

The cells may be isolated cells, e.g. they are not situated in a living animal or mammal. Preferably, the cell is a eukaryotic cell, more preferably a mammalian cell. Examples of mammalian cells include those from any organ or tissue from humans, mice, rats, hamsters, monkeys, rabbits, donkeys, horses, sheep, cows and apes. Preferably, the cells are human cells. The cells may be primary or immortalised cells. Preferred cells include HEK-293, HEK 293T, HEK-293E, HEK-293 FT, HEK-293S, HEK-293SG, HEK-293 FTM, HEK-293SGGD, HEK-293A, MDCK, C127, A549, HeLa, CHO, mouse myeloma, PerC6, 911 and Vero cell lines. Most preferably, the human cells are HEK293, HEK293T, HEK293A, PerC6 or 911. Other preferred cells include Hela, CHO and VERO cells. In some embodiments, the cells are induced pluripotent stem cells (iPS cells). In other embodiments, the cells are cancer cells.

The cell genome may be the cell's nuclear genome (e.g. one of the cell's chromosomes), the cell's mitochondrial DNA, plastid DNA, plasmid DNA or vector DNA, as desired. Preferably, the target site will be in chromosomal DNA.

As used herein, the term “introducing” one or more plasmids or vectors into the cell includes transformation, and any form of electroporation, conjugation, infection, transduction or transfection, inter alia. Viruses may be introduced into the cells by infection. Processes for such introduction are well known in the art (e.g. Proc. Natl. Acad. Sci. USA. 1995 Aug. 1; 92 (16):7297-301; and “Molecular Cloning: A Laboratory Manual” (Fourth Edition), Green, M R and Sambrook, J. (updated 2014)).

The one or more inhibitors may be introduced into the cells by any suitable means. For example, appropriate concentration(s) of inhibitors could be added directly into the cell culture medium of cells after the transfection/electroporation step.

The cells are cultured under conditions which promote the site-specific cleavage of the cell genome by the site-specific DNA endonuclease and the repair (preferably homology-directed repair) of the cleavage site(s) in the cell genome using the template DNA. Suitable culture conditions for cells are well known in the art (e.g. “Molecular Cloning: A Laboratory Manual” (Fourth Edition), Green, M R and Sambrook, J. (updated 2014)). In some embodiments, the cell will be present in a culture medium, preferably a liquid culture medium.

In another aspect, the invention provides a kit which may be used in the methods of the invention. In particular, the invention provides a kit comprising:
(a) AT9283 and one or more inhibitors selected from the group consisting of NU7441, S-PFI-2 and SMI-4a (preferably AT9283 and NU7441);

and optionally one or more of:
(i) a site-specific DNA endonuclease which is capable of producing a blunt-end double-stranded DNA cut in a cell genome, or a DNA plasmid or DNA vector encoding said endonuclease;
(ii) one or more guide RNAs, or a DNA plasmid or DNA vector encoding said guide RNAs; and
(iii) a template DNA molecule, or a DNA plasmid or DNA vector encoding said template DNA molecule.

The group may also comprise NU7026. The above components of the kit may be separate or one or more components may be mixed together.

In yet another embodiment, the invention provides a kit comprising:

(a) PHA-680632 and one or more inhibitors selected from the group consisting of NU7441, S-PFI-2 and SMI-4a (preferably PHA-680632 and NU7441);

and optionally one or more of:

(i) a site-specific DNA endonuclease which is capable of producing a blunt-end double-stranded DNA cut in a cell genome, or a DNA plasmid or DNA vector encoding said endonuclease;

(ii) one or more guide RNAs, or a DNA plasmid or DNA vector encoding said guide RNAs; and

(iii) a template DNA molecule, or a DNA plasmid or DNA vector encoding said template DNA molecule.

In yet another embodiment, the invention provides a kit comprising:

(a) TAK-901 and one or more inhibitors selected from the group consisting of NU7441, S-PFI-2 and SMI-4a (preferably TAK-901 and NU7441);

and optionally one or more of:

(i) a site-specific DNA endonuclease which is capable of producing a blunt-end double-stranded DNA cut in a cell genome, or a DNA plasmid or DNA vector encoding said endonuclease;

(ii) one or more guide RNAs, or a DNA plasmid or DNA vector encoding said guide RNAs; and

(iii) a template DNA molecule, or a DNA plasmid or DNA vector encoding said template DNA molecule.

The kit may also comprise one or more of AT9283, PHA-680632 and CCT137690.

In yet another embodiment, the invention provides a kit comprising:

(a) CCT137690 and one or more inhibitors selected from the group consisting of NU7441, S-PFI-2 and SMI-4a (preferably CCT137690 and NU7441);

and optionally one or more of:

(i) a site-specific DNA endonuclease which is capable of producing a blunt-end double-stranded DNA cut in a cell genome, or a DNA plasmid or DNA vector encoding said endonuclease;

(ii) one or more guide RNAs, or a DNA plasmid or DNA vector encoding said guide RNAs; and

(iii) a template DNA molecule, or a DNA plasmid or DNA vector encoding said template DNA molecule.

The kit may also comprise one or more of AT9283, PHA-680632 and TAK-901.

The groups may also comprise NU7026. The above components of the kits may be separate, or one or more components may be mixed together.

In yet another embodiment, the invention provides a kit comprising:

(a) AT9283 and PHA-680632, and one or more inhibitors selected from the group consisting of NU7441, S-PFI-2 and SMI-4a (preferably AT9283, PHA-680632 and NU7441);

and optionally one or more of:

(i) a site-specific DNA endonuclease which is capable of producing a blunt-end double-stranded DNA cut in a cell genome, or a DNA plasmid or DNA vector encoding said endonuclease;

(ii) one or more guide RNAs, or a DNA plasmid or DNA vector encoding said guide RNAs; and

(iii) a template DNA molecule, or a DNA plasmid or DNA vector encoding said template DNA molecule.

The group may also comprise NU7026. The group may also comprise TAK-901 and/or CCT137690. The above components of the kits may be separate, or one or more components may be mixed together.

The components of any of the kits may be separate, or one or more components may be mixed together.

The disclosure of each reference set forth herein is specifically incorporated herein by reference in its entirety.

### EXAMPLES

### Example 1

### Use of HEK293 Reporter Cell Line

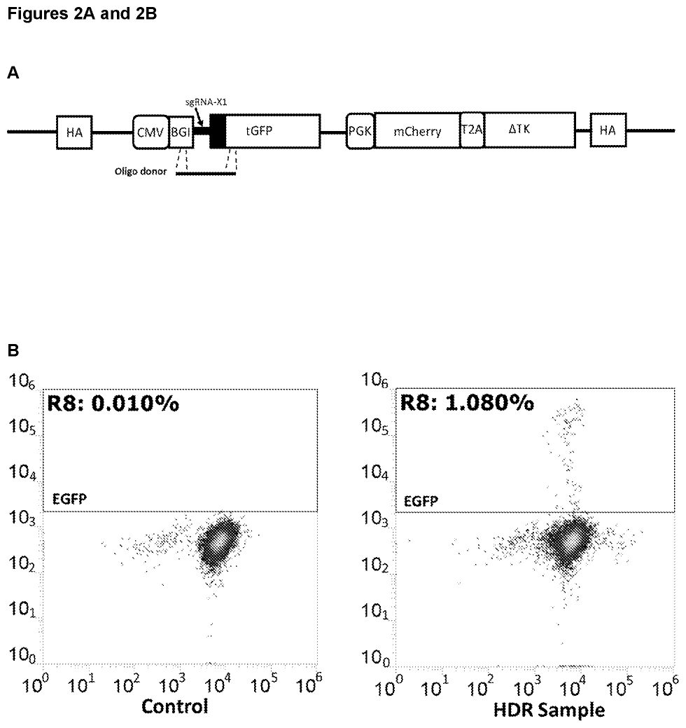

To investigate whether a knock-in truncated in HEK293 reporter cell line could be corrected by homology dependent repair, a CRISPR/Cas9-based HDR assay was used. We used ssODN as a donor template to correct the EGFP sequence and to restore functionality as ssODNs are known to be more efficient compared to the double-stranded donor for HDR based DNA repair. Briefly, cells were transfected with a wtCas9 ribonucleoprotein complex along with an oligo donor for restoring EGFP functionality. Cells were analysed by FACS 72 hours post-transfection (FIGS. 2A and 2B). The results indicated that, compared to the negative control and no-donor control, the EGFP expression was observed in the engineered HEK293-AAVS1_{(CMV-tEGFP-PGK-mCherry-ΔTK)} cell line transfected with wtCas9 ribonucleoprotein complex along with oligo donor. These results of HDR assay indicated that the HDR events using HEK293-AAVS1_{(CMV-tEGFP-PGK-mCherry-ΔTK)} cell line could be observed and quantified.

### Example 2

### Effect of Small Molecules on Cell Viability

To identify small molecule inhibitors which could increase the HDR efficiency, we used a small molecule library. To investigate the effect of this library on HDR efficiency and to identify novel molecules which would increase the HDR mediated gene editing efficiency, first we carried out a cell-viability assay using the HEK reporter cell line and alamar blue reagent. The experiment was performed to rule out any toxicity associated with the small molecule library. Briefly, 3 different concentration of inhibitors (0.1 μM, 1 μM and 10 μM) were added in the cells in a 96 well plate format and plates were incubated for 72 hours. After 72 hours, the media were replaced with alamar blue containing media and the plates were further incubated for 3 hours and then read on a Fluorstar omega plate reader. A varying range of effects was observed with different inhibitors as shown in the table below. The table gives the results obtained with the subsequently-selected inhibitors, together with a range of other potential inhibitors.

| S. | | % survival | | |
|---|---|---|---|---|
| No | Inhibitor | 0.1 μM | 1 μM | 10 μM |
| 1 | | 111.02 ± 3.54 | 112.35 ± 3.99 | 80.3 ± 4.54 |
| 2 | | 80.99 ± 1.01 | 15.56 ± 2.26 | 2.25 ± 0.31 |
| 3 | NU7441 | 103.58 ± 4.44 | 88.88 ± 3.55 | 22.95 ± 0.98 |
| | (KU-57788) | | | |
| 4 | | 103.3 ± 4.49 | 102.45 ± 3.22 | 100.51 ± 2.91 |
| 5 | | 104.58 ± 3.2 | 107.67 ± 0.62 | 108.88 ± 0.85 |
| 6 | SMI-4a | 105.2 ± 0.07 | 103.18 ± 5.38 | 99.85 ± 1.22 |
| 7 | | 100.52 ± 2.05 | 100.86 ± 1.83 | 95.18 ± 2.27 |
| 8 | | 103.84 ± 4.67 | 101.6 ± 2.07 | 103.31 ± 2.41 |
| 9 | NU7026 | 100.08 ± 4.01 | 100.14 ± 4.13 | 83.17 ± 1.44 |
| 10 | | 108.24 ± 1.3 | 94.46 ± 5.09 | 1.05 ± 0.1 |
| 11 | | 98.07 ± 2.25 | 39.52 ± 1.41 | 4.44 ± 0.67 |
| 12 | | 108.15 ± 1.35 | 100.58 ± 5.96 | 48.4 ± 2.77 |
| 13 | | 8.31 ± 0.06 | 7.21 ± 0.89 | 7.21 ± 0.09 |
| 14 | | 102.58 ± 0.88 | 101.51 ± 4.1 | 96.42 ± 0.64 |
| 15 | | 102.45 ± 0.9 | 102.92 ± 0.42 | 101.44 ± 8.52 |
| 16 | | 102.62 ± 0.68 | 98.02 ± 1.05 | 40.4 ± 0.04 |
| 17 | | 100.35 ± 1.4 | 103.27 ± 0.32 | 87.94 ± 9.49 |
| 18 | AT9283 | 81.49 ± 3.59 | 69.69 ± 1.2 | 7.1 ± 0.06 |
| 19 | S-PFI-2 | 99.98 ± 3.25 | 115.11 ± 2.47 | 94.25 ± 8.54 |
| 20 | | 105.84 ± 0.05 | 107.75 ± 6.83 | 107.43 ± 0.21 |
| 21 | | 93.75 ± 3.51 | 101.74 ± 3.28 | 74.49 ± 6.81 |
| 22 | | 94.34 ± 1.23 | 104.69 ± 1.85 | 84 ± 5.89 |
| 23 | | 93.88 ± 0.53 | 106.32 ± 0.4 | 74.51 ± 0.88 |
| 24 | TAK-901 | 88.9 ± 1.18 | 36.24 ± 0.88 | 5.65 ± 0.08 |
| 25 | CCT137690 | 105.48 ± 3.94 | 63.37 ± 2.12 | −0.08 ± 0.1 |

This Example demonstrates that it is not possible to use all potential inhibitors of double-stranded break repair mechanisms due to the inherent toxicity of some inhibitors. The inhibitor concentration permitting 75% cell survival was selected for HDR assays in subsequent screening.

### Example 3

### Effect of Small Molecules on HDR Efficiency in wtCas9 Induced Double Stranded Breaks

To identify the small molecules which would increases the HDR efficiency, we carried out experiments using the engineered HEK293 reporter cell line, ss-donor and wtCas9 in the presence of the inhibitors identified in Example 2. Briefly, in vitro reconstituted wtCas9 RNP with gRNA-X1 was transfected into the cells along with ssOligo donor (50 nM) in presence of the inhibitors. Inhibitor containing media was replaced with fresh media after 24 hours and cells were further incubated for 48 hours. After 48 hours cells were trypsinized and resuspended in 10% FBS containing PBS and analysed by FACS. As shown in FIGS. 2A and 2B, EGFP positive cells represent successful HDR events. A range of EGFP-positive cells were observed in the presence of the inhibitors. To identify compounds which increase the HDR efficiency, data was plotted as fold change compare to HDR efficiency in presence of Cas9 alone. Any compound exhibiting≥20% increase (˜fold change≥1.2) was selected to be a positive influencer of HDR event.

Only 5 compounds were observed to increase the HDR efficiency in the initial screen with wtCas9. Out of the 5 hit compounds, 2 were previously known inhibitors: NU7441 and NU7026 which target DNA-PKcs and inhibit NHEJ, and by doing so reciprocally increase the HDR efficiency. The other 3 inhibitors, S-PFI-2, SMI-4a and AT9283 have not been previously reported to be associated with increasing the HDR efficiency. These results suggest that these 3 molecules increase HDR efficiency upon introduction of double-stranded breaks.

To understand whether increase in HDR is related to dose response, we carried out the HDR assay using three different concentrations of selected inhibitors as shown in the table below.

| | Concentration tested | | | |
|---|---|---|---|---|
| Inhibitors | | Low | Medium | High |

| | | | | | | |
|---|---|---|---|---|---|---|
| S-PFI-2 | 5 | μM | 10 | μM | 20 | μM |
| NU7441 | 0.5 | μM | 1 | μM | 2 | μM |
| SMI-4a | 5 | μM | 10 | μM | 20 | μM |
| AT9283 | 0.05 | μM | 0.1 | μM | 0.2 | μM |

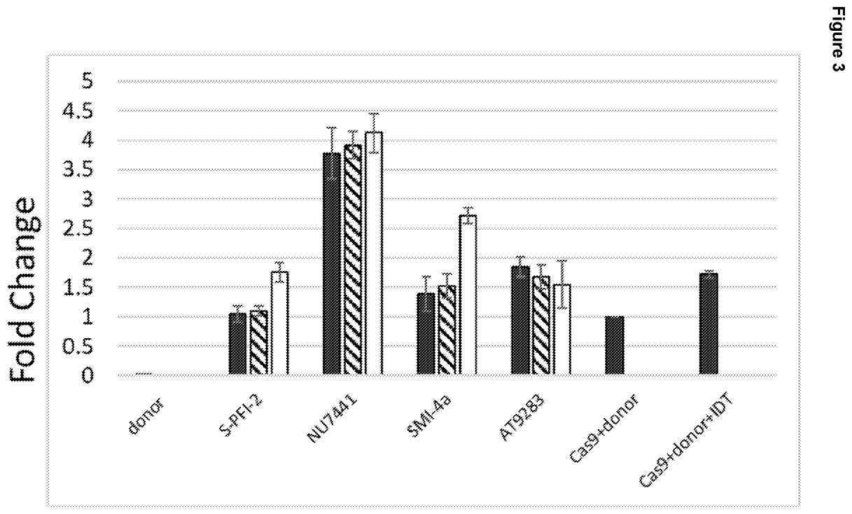

Low concentration is depicted by black bar, medium concentration is depicted by striped bar and high concentration is depicted by white bar in FIG. 3. Further, as shown in FIG. 3, S-PFI-2 and SMI-4a exhibited dose-dependent increases in HDR efficiency with the highest efficiency at 20 μM concentration, while AT9283 showed moderated decreases in HDR efficiency at a concentration of 0.2 μM. The highest HDR efficiency was observed with NU7441 and it exhibited moderate increases at a concentration of 2 μM. Optimal concentrations of S-PFI-2 (20 μM), NU7441 (1 μM), SMI4a (20 μM) and AT9283 (0.05 μM) were selected for further experiments.

### Example 4

### Effect of Small Molecule Combination on HDR

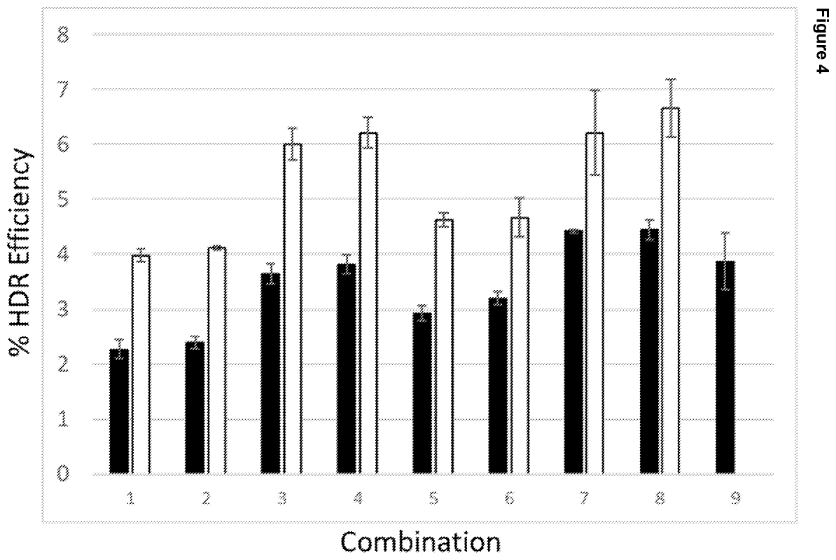

To investigate whether HDR efficiency would increase further by using the top hit small molecule combinations, we performed experiments using small molecule combinations for wtCas9. These combinations were selected using Design of Experiment (DoE) software. The results of small molecule inhibitor combinations using wtCas9 are shown in FIG. 4. These combination experiments were carried out in 2 sets: the first set was performed in the absence of NU7441 (a known HDR enhancer); and the second set was performed in the presence of NU7441. In first set of experiments, an increase in HDR efficiency was observed with (S)-PFI-2, AT9283, NU7441 and SMI-4a (as seen in previous experiments). When used in combination, all combinations showed increases in HDR efficiency compared to no compound (FIG. 4). In the second set of experiments, in the presence of NU7441, further increases (˜2fold) in HDR efficiency were observed compared to no NU7441 (FIG. 4). This effect of NU7441 was also apparent in other combinations as shown in FIG. 4.

Small molecule combinations without NU7441 (shown in FIG. 4 as black bars):

| Exp No | S-PF1 | AT9283 | SMI4a | NU7441 |
|---|---|---|---|---|
| 1 | No | No | No | No |
| 2 | Yes | No | No | No |
| 3 | No | Yes | No | No |
| 4 | Yes | Yes | No | No |
| 5 | No | No | Yes | No |
| 6 | Yes | No | Yes | No |
| 7 | No | Yes | Yes | No |
| 8 | Yes | Yes | Yes | No |

| | |
|---|---|
| 9 | IDT ENHANCER |

Small molecule combinations with NU7441 (shown in FIG. 4 as white bars):

| Exp No | S-PF1 | AT9283 | SMI4a | NU7441 |
|---|---|---|---|---|
| 1 | No | No | No | Yes |
| 2 | Yes | No | No | Yes |
| 3 | No | Yes | No | Yes |
| 4 | Yes | Yes | No | Yes |
| 5 | No | No | Yes | Yes |
| 6 | Yes | No | Yes | Yes |
| 7 | No | Yes | Yes | Yes |
| 8 | Yes | Yes | Yes | Yes |

| | |
|---|---|
| 9 | IDT ENHANCER |

### Example 5

### Effect of Aurora Kinase Inhibitor on HDR Efficacy

To investigate whether HDR efficiency is affected by the use of a different aurora kinase inhibitor, we compared the following combinations:
1. AT9283, (S)-PFI-2, NU7441 and SMI-4a
2. PHA-680632 (1μM), (S)-PFI-2, NU7441 and SMI-4a.

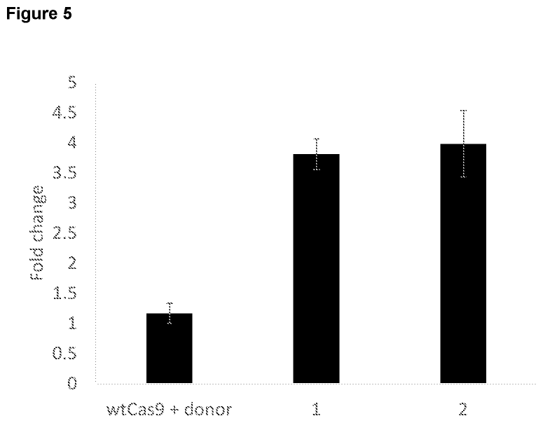

Both combinations showed similar increases in HDR efficiency compared to a no- compound control (FIG. 5).

### REFERENCES

1. Bunting et al. (2010) 53BP1 Inhibits Homologous Recombination in Brca1-deficient Cells by Blocking Resection of DNA Breaks, Cell, 141(2):243-54

2. Escribano-Diaz et al. (2013) DNA repair pathway choice—a PTIP of the hat to 53BP1, EMBO reports, 14(8): 665-666

3. Zimmermann et al. (2013) 53BP1 Regulates DSB Repair Using Rif1 to Control 5′ End Resection, Science, 339(6120):700-4

4. Fradet-Turcotte et al. (2013) 53BP1 Is a Reader of the DNA-damage-induced H2A Lys 15 Ubiquitin Mark, Nature, 499(7456):50-4

5. Sartori et al. (2007) Human CtIP Promotes DNA End Resection, Nature, 450(7169):509-14

6. Symington et al. (2011) Double-strand Break End Resection and Repair Pathway Choice, Annu Rev Genet, 45:247-71

7. Symington et al. (2016) Mechanism and Regulation of DNA End Resection in Eukaryotes, Crit Rev Biochem Mol Biol., 51(3): 195-212

8. Zakharyevich et al. (2010) Temporally and biochemically distinct activities of Exo1 during meiosis: double-strand-break resection and resolution of double-Holliday Junctions, Mol Cell, 40(6): 1001-1015

9. Jinek, M. et al. (2012), A Programmable dual-RNA-guided DNA Endonuclease in Adaptive Bacterial Immunity, Science, 337(6096):816-21

10. Briner, A. E. et al. (2014). “Guide RNA functional modules direct cas9 activity and orthogonality”. Molecular Cell, 56(2), 333-339

1. A method for promoting the modification of a target site in a genome of a cell,
the method comprising the steps of introducing:
(i) a template DNA molecule which has DNA sequence homology with the target site; and
(ii) one or more inhibitors;
into a cell which comprises or is capable of expressing a site-specific DNA endonuclease, thereby promoting the site-specific cleavage of the cell genome by the site-specific DNA endonuclease and the modification of the target site in the cell genome,
wherein the one or more inhibitors comprise an aurora kinase inhibitor selected from the group consisting of AT9283, PHA-680632, TAK-901 and CCT137690, and wherein the site-specific endonuclease is one which produces a blunt-end double-stranded cut in the cell genome.

2. The method as claimed in claim 1, wherein the one or more inhibitors comprise AT9283.

3. The method as claimed in claim 1, wherein the one or more inhibitors comprise:
(a) PHA-680632, or
(b) AT9283 and PHA-680632.

4. The method as claimed in claim 1, wherein the one or more inhibitors comprise TAK-901 and/or CCT137690.

5. The method as claimed in claim 1, wherein the site-specific DNA endonuclease is an RNA-guided endonuclease, or a CRISPR RNA-guided endonuclease, and one or more CRISPR gRNAs are additionally introduced into the cell.

6. The method as claimed in claim 1, wherein the CRISPR RNA-guided endonuclease is a Type II CRISPR system enzyme.

7. The method as claimed in claim 6, wherein the CRISPR endonuclease which produces a blunt double-stranded cut in the cell genome is Cas9.

8. The method as claimed in claim 7, wherein the Cas9 endonuclease is derived or obtained from S. pneumoniae, S. pyogenes, or S. thermophilus Cas9, or is a variant thereof.

9. The method as claimed in claim 1 wherein the inhibitors comprise AT9283 and/or PHA-680632, together with one or more additional inhibitors selected from the group consisting of NU7441, S-PFI-2 and SMI-4a.

10. The method as claimed in claim 9, wherein the inhibitors comprise:
AT9283, NU7441 and S-PFI-2;
AT9283, NU7441 and SMI-4a;
AT9283, NU7441, S-PFI-2 and SMI-4a;
AT9283, NU7026 and S-PFI-2;
AT9283, NU7026 and SMI-4a; or
AT9283, NU7026, S-PFI-2 and SMI-4a.

11. The method as claimed in claim 9, wherein the inhibitors comprise:
PHA-680632, NU7441 and S-PFI-2;
PHA-680632, NU7441 and SMI-4a;
PHA-680632, NU7441, S-PFI-2 and SMI-4a;
PHA-680632, NU7026 and S-PFI-2;
PHA-680632, NU7026 and SMI-4a; or
PHA-680632, NU7026, S-PFI-2 and SMI-4a.

12. The method as claimed in claim 9, wherein the inhibitors comprise:
AT9283, PHA-680632, NU7441 and S-PFI-2;
AT9283, PHA-680632, NU7441 and SMI-4a;
AT9283, PHA-680632, NU7441, S-PFI-2 and SMI-4a;
AT9283, PHA-680632, NU7026 and S-PFI-2;
AT9283, PHA-680632, NU7026 and SMI-4a; or
AT9283, PHA-680632, NU7026, S-PFI-2 and SMI-4a.

13. The method as claimed in claim 1 wherein the inhibitors comprise TAK-901 and/or CCT137690, together with one or more additional inhibitors selected from the group consisting of NU7441, S-PFI-2 and SMI-4a.

14. The method as claimed in claim 13, wherein the inhibitors comprise:
TAK-901, NU7441 and S-PFI-2;
TAK-901, NU7441 and SMI-4a;
TAK-901, NU7441, S-PFI-2 and SMI-4a;
TAK-901, NU7026 and S-PFI-2;
TAK-901, NU7026 and SMI-4a; or
TAK-901, NU7026, S-PFI-2 and SMI-4a.

15. The method as claimed in claim 13, wherein the inhibitors comprise:
CCT137690, NU7441 and S-PFI-2;
CCT137690, NU7441 and SMI-4a;
CCT137690, NU7441, S-PFI-2 and SMI-4a;
CCT137690, NU7026 and S-PFI-2;
CCT137690, NU7026 and SMI-4a; or
CCT137690, NU7026, S-PFI-2 and SMI-4a

16. The method as claimed in claim 1, wherein the cell is a mammalian cell, or a human cell.

17. A kit comprising:
(a) AT9283,
(b) PHA-680632, or
(c) AT9283 and PHA-680632,
and one or more inhibitors selected from the group consisting of NU7441, NU7026, S-PFI-2 and SMI 4a,
and optionally one or more of:
(i) a site-specific DNA endonuclease which is capable of producing a blunt-end double-stranded cut in a cell genome, or a DNA plasmid or DNA vector encoding said endonuclease;
(ii) one or more guide RNAs, or a DNA plasmid or DNA vector encoding said guide RNAs; and
(iii) a template DNA molecule, or a DNA plasmid or DNA vector encoding said template DNA molecule.

18. A kit comprising:
TAK-901 and/or CCT137690, and one or more inhibitors selected from the group consisting of NU7441, NU7026, S-PFI-2 and SMI-4a,
and optionally one or more of:
(i) a site-specific DNA endonuclease which is capable of producing a blunt-end double-stranded cut in a cell genome, or a DNA plasmid or DNA vector encoding said endonuclease;
(ii) one or more guide RNAs, or a DNA plasmid or DNA vector encoding said guide RNAs; and
(iii) a template DNA molecule, or a DNA plasmid or DNA vector encoding said template DNA molecule.

19. The kit as claimed in claim 17, wherein the one or more inhibitors is selected from the group consisting of AT9283 and NU7441.
